(19) United States
(12) Patent Application Publication
(10) Pub. No.: US 2024/0408357 A1
(43) Pub. Date: Dec. 12, 2024

(54) IV Catheter Securement Device

(71) Applicant: Quest Medical, Inc., Allen, TX (US)

(72) Inventors: Robert Mart, Colleyville, TX (US); Jeffrey Albertsen, McKinney, TX (US)

(21) Appl. No.: 18/736,721

(22) Filed: Jun. 7, 2024

Related U.S. Application Data
(60) Provisional application No. 63/471,841, filed on Jun. 8, 2023.

(51) Int. Cl. A61M 25/02

(52) U.S. Cl. CPC A61M 25/02; A61M 2025/0266

(57) ABSTRACT

An embodiment includes a custom-forming peripheral intravenous (PIV) catheter stabilization system that surrounds and conforms to the PIV catheter hub and/or a luer fitting and/or a needleless connector (NC) administration set connection. The embodiment provides a uniquely stable, energy absorbing primary intravenous (IV) stabilization system.

### CROSS-REFERENCE TO RELATED APPLICATION

This application claims priority to U.S. Provisional Patent Application No. 63/471,841 filed on Jun. 8, 2023 and entitled “IV Catheter Securement Device”, the content of which is hereby incorporated by reference.

### BACKGROUND

A Vascular Access Device (VAD) includes any device utilized for venous access regardless of location. VADs include, for example, peripheral intravenous (PIV) catheters, peripherally inserted central (PIC) catheters, central venous (CV) catheters, and implanted venous ports.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 includes a side view of an embodiment.

FIG. 2 include a top view of an embodiment.

FIG. 3A includes a perspective view of an embodiment with an open configuration.

FIG. 3B includes a perspective view of an embodiment with an open configuration.

FIG. 4A includes a perspective view of an embodiment with an open configuration.

FIG. 4B includes a perspective view of an embodiment with an open configuration.

FIG. 5 includes a perspective view of an embodiment with an open configuration.

FIG. 6 includes a perspective view of an embodiment with an open configuration.

FIG. 7 includes a side view of an embodiment.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

Applicant determined forty percent of VAD catheters fail due to complications and that stabilizing VAD catheters within a vein is critical to avoid or limit those complications. Further, Applicant determined securely holding the VAD in place will minimize complications by limiting pistoning of the device (e.g., in and out movements and/or back and forth movements of the device within a vessel wall). Minimizing catheter movement inside the vein minimizes endothelial cell irritation/disruption. Maintaining skin integrity is also critical as skin injury contributes to VAD failure.

Put another way, Applicant determined stabilizing a VAD catheter within a vein is key in avoiding or limiting complications and that securing the catheter, hub, and IV line is key in stabilizing the catheter within a vein. Movement or tension on the IV line can put pressure on the VAD, resulting in catheter movement or migration, which in turn may result in complications. Despite varied conventional products (ranging from simple tape to complex medical devices) and dedicated nursing attention, there remains a high VAD failure rate. Overall, IV catheter failure rate lies between 35% and 50%.

However, an embodiment addresses these issues by providing a Manufactured Catheter Securement Device (MCSD). The embodiment includes a custom-forming PIV catheter stabilization system that surrounds and conforms to the PIV catheter hub and/or a luer fitting and/or a needleless connector (NC) administration set connection. The embodiment provides a uniquely stable, energy absorbing primary IV stabilization system.

An embodiment provides a custom-forming PIV catheter hub/luer fitting stabilization system that surrounds and conforms to the PIV catheter hub and/or luer administration set connection to provide a stable, energy absorbing primary IV stabilization system. The embodiment includes top and bottom gel pads that form a “sandwich” to secure the catheter system, whereby each pad is secured by adhesive-backed breathable fabric. The gel pads are stable (e.g., do not dry out) and may be tacky (e.g., silicone gel adhesive). The gel pads may include an anhydrous gel. The gel may include, for example, silicone, elastomeric, foam, or any of a number of stable but conformable materials such as putties, hydrocolloids, and the like. A lower surface of the stabilization device includes a wedge-shaped surface so the catheter and catheter hub are held at approximately the same angle as the catheter insertion through the skin and into the vessel lumen. This minimizes tension on the catheter and helps ensure the lumen does not distort or kink and to minimizes patient discomfort.

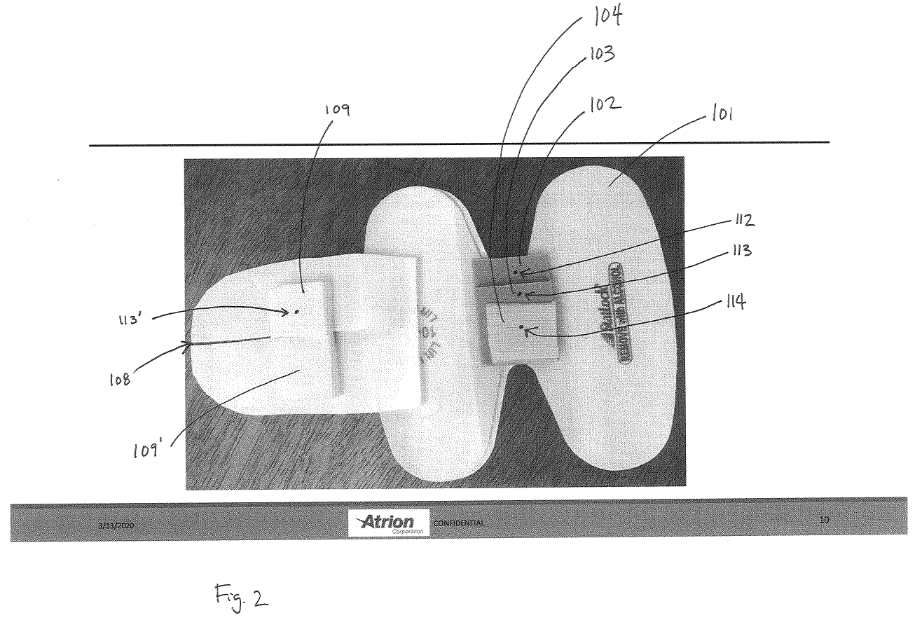

An embodiment includes a self-forming IV catheter/hub/luer PIVC securement system that conforms to the IV catheter hub/male luer administration set connection to provide a stable, energy absorbing, primary IV securement system. See, for example, FIG. 2. The system consists of top and bottom conforming pads that form a sandwich around the hub/luer fitting connection. Each pad is secured by an adhesive backed substrate (e.g., a breathable fabric). These conforming, energy absorbing pads can be comprised of a gel (e.g., an anhydrous gel including, for example, silicone, elastomeric, foam, or any of a number of stable but conformable materials such as putties, hydrocolloids, and the like). A pad that has some level of tack is also advantageous as it better secures the catheter hub/luer and is used in some embodiments.

In some embodiments, the bottom conforming pad is shaped as a wedge that is thinner on the catheter hub side and thicker on the IV tubing side. This maintains the entry angle of the catheter, which helps to prevent catheter kinking (which could lead to occlusion) and reduces catheter pressure on tissue that may cause irritation and patient discomfort. This entry angel wedge pad can be comprised of a single wedge of material (e.g., a stable gel, foam, or pad) or can be formed by stacking at least two layers of material in a step-wise fashion.

An alternative configuration includes only having the bottom, wedge shaped pad and adhesive backed carrier with the top layer being just an adhesive backed strip (e.g., tape), that covers the catheter hub.

Because the catheter hub/luer connectors are form-fitted into the pad, the pad holds the hub/luer connector firmly in place to help prevent the catheter form being prematurely pulled out of a vessel. It also reduces “pistoning” which is the movement of the catheter forward and backward inside a blood vessel, and lateral movement of catheter along the vessel walls. Both of these actions can irritate or disrupt the endothelial cell lining of the vessel which can result in inflammation leading to infiltration and other catheter complications which may necessitate catheter removal.

In the above configurations, the top layer can be a separate strip or strips, or it can be directly connected to the bottom adhesive carrier and then folded over to cover the catheter hub/luer connection.

Embodiments have numerous advantages over convention systems. For example, embodiments: (1) automatically conform (due to soft and pliable design) to various catheter hubs and/or luer fittings and/or NCs to provide stable, primary stabilization of PICV administration sets to patients, (2) reduce pressure, tension, and movement on VADs (reducing the risk of VAD catheter complications), (3) help provide safe mobility for patients, (4) lessen needs for additional/excessive tape to secure catheter systems, (5) are appropriate in numerous and diverse clinical environments (e.g., hospital, home, ambulatory surgery centers, emergency/transport vehicles).

PIVC catheter securement is a use for embodiments. However, other vascular devices (e.g., PIC or CV catheters) are alternative uses. Other potential applications include perfusing catheters (e.g., g tubes) or any fluid collection catheter (e.g., urinary catheters).

### Example Set

Example 1 includes an intravenous (IV) line securement system (100). The system comprises a first layer (101) including first and second opposing surfaces, wherein the first surface of the first layer couples to an adhesive that is configured to couple the first layer to a patient's skin. The system comprises a second layer (102) including first and second opposing surfaces, wherein: (a) the first surface of the second layer is fixedly coupled to the second surface of the first layer, and (b) the second layer is more rigid than the first layer. The system comprises a third layer (103) including first and second opposing surfaces, wherein: (a) the first surface of the third layer is fixedly coupled to the second surface of the second layer, (b) the third layer is less rigid than the second layer, (c) the second surface of the third layer couples to an adhesive (104) that is configured to couple to the IV line, (d) the second surface of the third layer includes a channel configured to receive the IV line, (e) the first surface of the third layer is primarily disposed in a first plane (106), and (f) a second plane (131), parallel to the first plane, intersects the third layer and the channel.

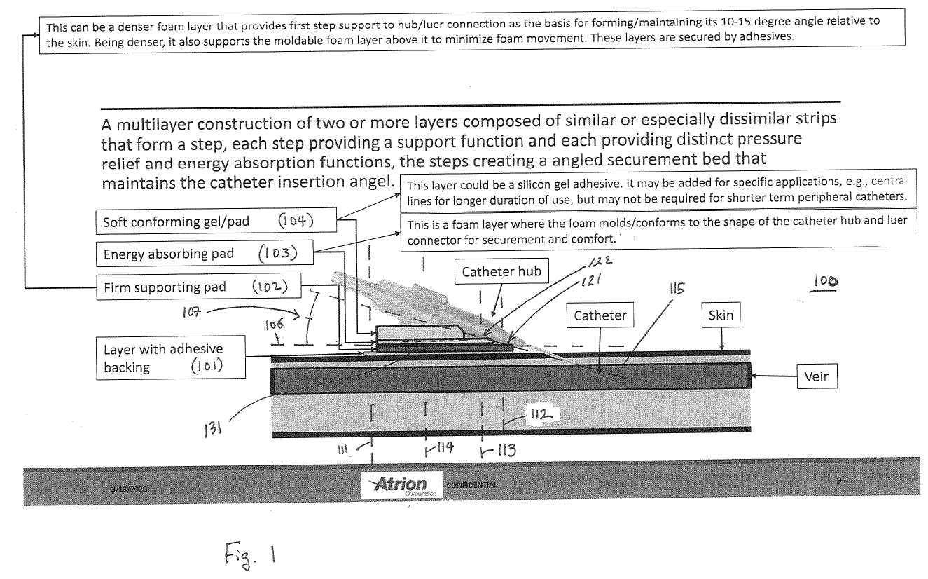

For example, see FIGS. 1 and 2. FIG. 1 provides a multilayer construction including two or more layers (which may be similar or dissimilar to one another in terms of material composition, density, rigidity, and the like) that collectively form a step (or at least one step). One or more of the steps provide a support function, pressure relief, and/or energy absorption functions. The steps create an angled securement bed that maintains the catheter insertion angle into the patient.

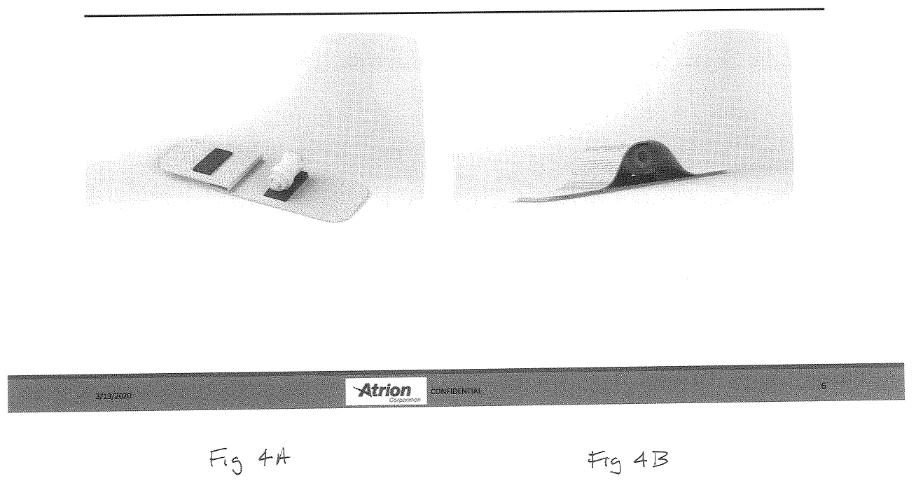

Layer 101 may include a fabric layer with an adhesive backing. The layer may be long enough to wrap around the other layers to “sandwich” the IV line components between the upper and lower portions of the first layer. FIG. 4B shows an example of such a “sandwich” effect. The first layer may include a monolithic length of material or may be composed of multiple portions that are stitched or adhered to one another. In FIG. 2 the first layer is composed of multiple portions that are not monolithic with each other. FIG. 2 provides an embodiment whereby the upper or top portion of layer 101 is split at area 108 to provide flexibility for the layer 101 to secure portions of the catheter system having differing diameters or distances from the skin.

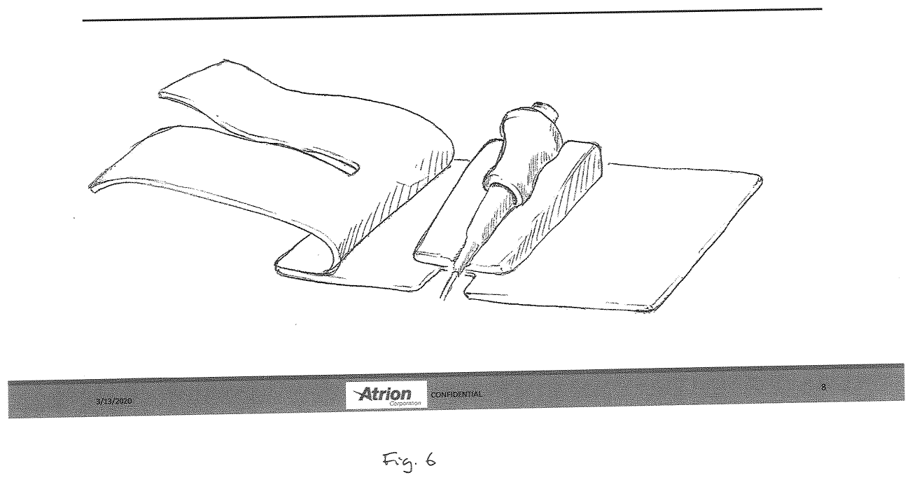

FIG. 6 includes another embodiment including a split top layer.

Layer 102 may include a gel or foam layer that is denser than layer 101 and which provides a step support to the hub and/or luer connection as the basis for forming and maintaining a 10-15 degree angle (see angle 107) relative to the skin. Being denser, layer 102 also supports the moldable gel or foam layer (layer 103) to minimize gel or foam movement. Layers 101, 102, 103 may be secured to one another using adhesives. For other non-VAD applications (e.g., fluid collection), an angled gel/pad may not be required.

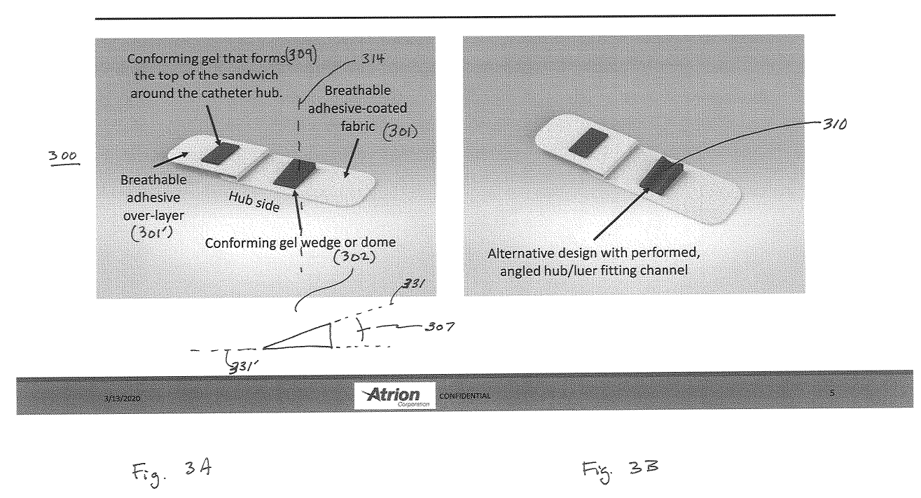

Layer 103 includes a gel or foam layer where the gel or foam molds or conforms to the shape of the catheter hub and/or luer connector for securement of the device and comfort for the patient. While not shown in FIG. 1 or 2, a representative channel is shown in FIG. 3B. Layer 103 acts as a shock absorber, absorbing energy and vibrations while minimizing the transfer of energy to the catheter (wherein catheter movement inside the vein is a major cause of inflammation which can lead to catheter failure).

Layer 104 may include a silicon gel adhesive. Layer 104 may be included in specific applications (e.g., central lines for longer duration of use) but omitted for shorter term catheter applications. Additional stacked layers may also be added as needed (e.g., to increase the wedge angle for catheters inserted into deeper vessels).

As used herein, two elements “fixedly coupled” to each other accounts for elements directly contacting each other or indirectly contacting each other (e.g., via one or more sublayers).

As used herein, the “IV line” includes catheters, conduits, connectors (e.g., luer fitting), hubs, and the like.

Example 2. The IV line securement system of Example 1, wherein: a second axis (112), orthogonal to the first plane, intersects the first and second layers but not the third layer; and a third axis (113), orthogonal to the first plane, intersects the first, second, and third layers.

Example 3. The IV line securement system of Example 2, wherein: in a system open orientation the third axis intersects the first layer only one time; and in a system closed orientation the third axis intersects the first layer two times respectively at first and second locations. The second and third layers are between the first and second locations.

For example, in FIG. 2 point 113′ indicates where axis 113 will intersect the upper strip of material in the “closed position”. Point 113′ is shown on padding but may be directly in line with axis 113 as the axis intersects the upper surface of the upper strip of material.

FIG. 4A is an example of a “system opened orientation” and FIG. 4B is an example of a “system closed orientation”.

Example 3.1 The IV line securement system of Example 3 comprising a compliant pad (109) connected to the first layer at the second location.

For example, see FIG. 2.

Example 3.2 The IV line securement system of Example 3 comprising at least one compliant pad connected to the first layer, wherein in the system closed orientation the at least one compliant pad and the third layer are configured to sandwich a portion of the IV line between the at least one compliant pad and the third layer.

See, for example, pad 109 and/or pad 109′ of FIG. 2.

Example 4. The IV line securement system of Example 3, wherein the first and second locations are included on a monolithic portion of the first layer.

As used herein, “monolithic” means formed of a single uninterrupted material without seams, stiches, adhesives and the like being used to join two elements to one another.

Example 5. The IV line securement system of Example 2, wherein a first axis (111), orthogonal to the first plane, intersects the first layer but not the second layer and not the third layer.

Example 6. The IV line securement system of Example 2, wherein: a fourth axis (114), orthogonal to the first plane, intersects the first, second, and third layers and the adhesive coupled to the second surface of the third layer. In a system open orientation the fourth axis intersects the first layer only one time. In a system closed orientation the fourth axis intersects the first layer two times respectively at first and second locations. The second and third layers are between the first and second locations.

Example 7. The IV line securement system of Example 6, wherein the first and second locations are included on a monolithic portion of the first layer.

Example 8. The IV line securement system according to any of Examples 2-8, wherein a portion (121) of the second surface of the second layer is not covered by the third layer. A portion (122) of the second surface of the third layer is not covered by the adhesive on the second surface of the third layer. Collectively the portions of the second surfaces of each of the second and third layers form a stair-step profile.

Example 9. The IV line securement system of Example 8, wherein: a fifth axis (115) intersects each of the portions 121, 122 of the second surfaces of each of the second and third layers. The fifth axis intersects the first plane at an angle (107) of between 0 to 20 degrees.

In another embodiment, the angle is between 10 and 15 degrees. In another embodiment, the angle is between 8 and 20 degrees.

Example 10. The IV line securement system according to any of Examples 1 to 9, wherein: the first layer includes no sublayers; the second layer includes no sublayers; and the third layer includes no sublayers.

However, in other embodiments any or all of the first, second, and third layers may include sublayers.

Alternative version of Example 1. An intravenous (IV) line securement system (100) comprising: a first layer (101) including first and second opposing surfaces, wherein the first surface of the first layer has an adhesive to couple the first layer to a patient's skin; a second layer (102) including first and second opposing surfaces, wherein: (a) the first surface of the second layer is fixedly coupled to the second surface of the first layer, and (b) the second layer is more rigid than the first layer; a third layer (103) including first and second opposing surfaces, wherein: (a) the first surface of the third layer is fixedly coupled to the second surface of the second layer, (b) the third layer is less rigid than the second layer, and (c) the second surface of the third layer has an adhesive (104) configured to couple to the IV line.

Thus, alternative embodiments may not necessarily include a channel within layer 103.

Alternative version of Example 1. An intravenous (IV) line securement system (100) comprising: a first layer (102) including first and second opposing surfaces, wherein the first surface of the first layer has an adhesive sublayer configured to couple the first layer to a patient's skin; a second layer (103) including first and second opposing surfaces, wherein: (a) the first surface of the second layer is fixedly coupled to the second surface of the first layer, (b) the second layer is less rigid than the first layer, (c) the second surface of the second layer has an adhesive (104) configured to couple to the IV line, (d) the second surface of the second layer includes a channel configured to receive the IV line, (e) the first surface of the second layer is primarily disposed in a first plane (106), and (f) a second plane, parallel to the first plane, intersects the second layer and the channel.

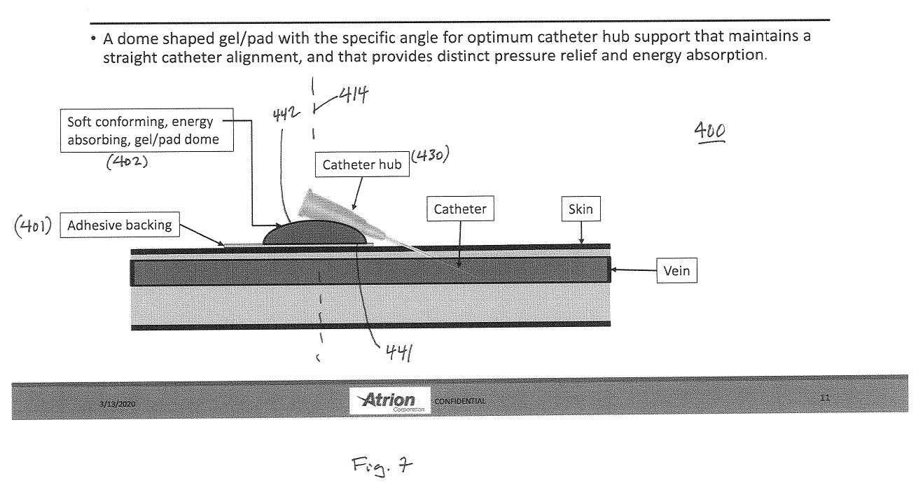

Thus, not all embodiments include more than two layers. See, for example, FIGS. 3 and 7. The second layer may not necessarily have adhesive 104 and/or a channel. For example, without a channel the second layer may have sufficient pliability to conform to the catheter system and thereby stabilize the system.

As used herein, an element that “has” an adhesive may include an adhesive layer or application on a surface of the element, or the element itself may be an adhesive material.

Example 1a. An intravenous (IV) line securement system (300) comprising a first layer portion (301) including first and second opposing surfaces, wherein the first surface of the first layer portion has an adhesive configured to couple the first layer portion to a patient's skin. The system includes a first compliant pad (302) including first and second opposing surfaces, wherein: (a) the first surface of the first compliant pad is fixedly coupled to the second surface of the first layer portion, (b) the first surface of the first compliant pad is included within a first plane (331′), (c) the second surface of the first compliant pad is included within a second plane (331), and (d) the first and second planes intersect one another at an angle (307) that is between 0 to 20 degrees. A second layer portion (301′) includes first and second opposing surfaces. The system includes a second compliant pad (309) including first and second opposing surfaces, wherein the first surface of the second compliant pad is fixedly coupled to the second surface of the second layer portion. The first and second layer portions are pivotably coupled to one another. In a system open orientation an axis (314) intersects the first layer portion and the first compliant pad but not the second layer portion and not the second compliant pad. In a system closed orientation the axis intersects the first layer portion, the first compliant pad, the second layer portion, and the second compliant pad.

In another embodiment, the first and second planes intersect one another at an angle (307) that is between 8 to 20 degrees;

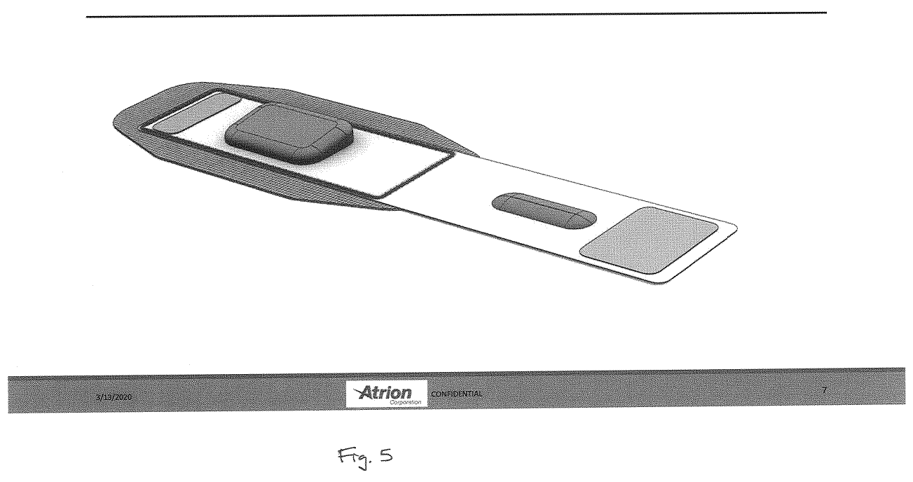

For example, see FIGS. 3A and 3B. As another example, see FIG. 5. The portions on the outer edges (outside the portions that will sandwich the hub/device) represent a fixation system where the breathable adhesive over layer (301′ of FIG. 3A) is folded over the catheter hub (as in FIG. 4B) to secure the top layer to the bottom layer (301). These portions may include fixation systems such as hook and loop systems, adhesives, and the like.

Example 2a. The IV line securement system of Example 1a, wherein the second surface of the first compliant pad includes a channel (310) configured to receive the IV line.

For example, see FIG. 3B.

Example 3a. The IV line securement system according to any of Examples 1a to 2a, wherein the first and second layer portions are monolithic with each other.

Example 4a. The IV line securement system according to any of Examples 1a to 3a, wherein in the system closed orientation the first and second compliant pads are configured to sandwich a portion of the IV line (330) between the first and second compliant pads.

For example, FIGS. 4A and 4B respectively illustrate system open and closed orientations.

Example 1b. An intravenous (IV) line securement system (400) comprising: a first layer portion (401) including first and second opposing surfaces, wherein the first surface of the first layer portion has an adhesive configured to couple the first layer portion to a patient's skin. The system includes a first compliant pad (402) including first and second opposing surfaces, wherein: (a) the first surface (441) of the first compliant pad is fixedly coupled to the second surface of the first layer portion, (b) the first surface of the first compliant pad is included within a first plane and is predominantly planar, (c) the second surface (442) of the first compliant pad is domed and predominantly curvilinear. The system includes a second layer portion including first and second opposing surfaces. The system includes a second compliant pad including first and second opposing surfaces, wherein: (a) the first surface of the second compliant pad is fixedly coupled to the second surface of the second layer portion. The first and second layer portions are pivotably coupled to one another. In a system open orientation an axis (414) intersects the first layer portion and the first compliant pad but not the second layer portion and not the second compliant pad. In a system closed orientation the axis intersects the first layer portion, the first compliant pad, the second layer portion, and the second compliant pad.

For example, see FIG. 7 (which does not disclose the second layer portion but which is similar to FIG. 3A in that regard).

Example 2b. The IV line securement system of Example 1b, wherein the first and second layer portions are monolithic with each other.

Example 3b. The IV line securement system according to any of Examples 1b to 2b, wherein in the system closed orientation the first and second compliant pads are configured to sandwich a portion of the IV line (430) between the first and second compliant pads.

In an embodiment the channel for receiving the hub (or component coupled to the hub) includes a deviation or ridges so a user knows how far up the hub (or related component) should be in the channel. For example, in FIG. 1 if the luer fitting were further to the right it would change the angle at which the needle is in the vessel. While the exact level or location at which the hub (or related component) interfaces the securement system may not be critical in some applications, in others it may be more important and used to more directly manage the interface between the IV system and the patient. For example, a ridge on the channel may be configured to align with a rigid on the hub (or component related there to). The interface may key the securement system/hub system to one another via a keyed interface. This may help standardize the angle at which, for example, a needle is inserted into a patient.

In other embodiments, the goal may be to provide more flexibility to the user and provide a channel that does not have deviations and the like (which deviations may hinder the ability of the user to select where to place the hub (or related component)). More generally, differences in anatomy and vein location may change the desired insertion angel. In such situations, the angles do not have to match exactly. Instead, the securement device just needs to minimize the deflection of, for example, the catheter as it exists the skin. Having flexibility to adjust to these differences in anatomy/desired insertion angle is a better clinical design than the rigid, fixed angle provided by conventional rigid systems.

Example 1c. An intravenous (IV) line securement system (100) comprising: a first layer (101) including first and second opposing surfaces, wherein the first surface of the first layer couples to an adhesive that is configured to couple the first layer to a patient's skin; a second layer (102) including first and second opposing surfaces, wherein the first surface of the second layer is fixedly coupled to the second surface of the first layer; a third layer (103) including first and second opposing surfaces, wherein: (a) the first surface of the third layer is fixedly coupled to the second surface of the second layer, (b) the second surface of the third layer couples to an adhesive (104) that is configured to couple to the IV line, (c) the second surface of the third layer includes a channel configured to receive the IV line, (d) the first surface of the third layer is primarily disposed in a first plane (106), and (e) a second plane (131), parallel to the first plane, intersects the third layer and the channel.

The embodiment of Example 1c is similar to the embodiment of Example 1 but does not necessarily require any differentials in density or stiffness or flexibility between the layers.

Example 2c. The IV line securement system of Example 1c, wherein: a second axis (112), orthogonal to the first plane, intersects the first and second layers but not the third layer; a third axis (113), orthogonal to the first plane, intersects the first, second, and third layers.

Example 3c. The IV line securement system of Example 2c, wherein: in a system open orientation the third axis intersects the first layer only one time; in a system closed orientation the third axis intersects the first layer two times respectively at first and second locations; the second and third layers are between the first and second locations.

Example 3.1c The IV line securement system of Example 3c comprising a compliant pad (109) connected to the first layer at the second location.

Example 3.2c The IV line securement system of Example 3c comprising at least one compliant pad connected to the first layer, wherein in the system closed orientation the at least one compliant pad and the third layer are configured to sandwich a portion of the IV line between the at least one compliant pad and the third layer.

Example 4c. The IV line securement system of Example 3c, wherein the first and second locations are included on a monolithic portion of the first layer.

Example 5c. The IV line securement system of Example 2c, wherein a first axis (111), orthogonal to the first plane, intersects the first layer but not the second layer and not the third layer.

Example 6c. The IV line securement system of Example 2c, wherein: a fourth axis (114), orthogonal to the first plane, intersects the first, second, and third layers and the adhesive coupled to the second surface of the third layer; in a system open orientation the fourth axis intersects the first layer only one time; in a system closed orientation the fourth axis intersects the first layer two times respectively at first and second locations; the second and third layers are between the first and second locations.

Example 7c. The IV line securement system of Example 6c, wherein the first and second locations are included on a monolithic portion of the first layer.

Example 8c. The IV line securement system according to any of Examples 2c-8c, wherein: a portion (121) of the second surface of the second layer is not covered by the third layer; a portion (122) of the second surface of the third layer is not covered by the adhesive on the second surface of the third layer; collectively the portions of the second surfaces of each of the second and third layers form a stair-step profile.

Example 9c. The IV line securement system of Example 8c, wherein: a fifth axis (115) intersects each of the portions of the second surfaces of each of the second and third layers; the fifth axis intersects the first plane at an angle (107) of between 0 to 20 degrees.

Example 10c. The IV line securement system according to any of Examples 1c to 9c, wherein: the first layer includes no sublayers; the second layer includes no sublayers; and the third layer includes no sublayers.

Alternative version of Example 1c. An intravenous (IV) line securement system (100) comprising: a first layer (101) including first and second opposing surfaces, wherein the first surface of the first layer has an adhesive to couple the first layer to a patient's skin; a second layer (102) including first and second opposing surfaces, wherein the first surface of the second layer is fixedly coupled to the second surface of the first layer; a third layer (103) including first and second opposing surfaces, wherein: (a) the first surface of the third layer is fixedly coupled to the second surface of the second layer, and (b) the second surface of the third layer has an adhesive (104) configured to couple to the IV line.

Alternative version of Example 1c. An intravenous (IV) line securement system (100) comprising: a first layer (102) including first and second opposing surfaces, wherein the first surface of the first layer has an adhesive sublayer configured to couple the first layer to a patient's skin; a second layer (103) including first and second opposing surfaces, wherein: (a) the first surface of the second layer is fixedly coupled to the second surface of the first layer, (b) the second surface of the second layer has an adhesive (104) configured to couple to the IV line, (c) the second surface of the second layer includes a channel configured to receive the IV line, (d) the first surface of the second layer is primarily disposed in a first plane (106), and (e) a second plane, parallel to the first plane, intersects the second layer and the channel.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. An intravenous (IV) line securement system comprising:
a first layer including first and second opposing surfaces, wherein the first surface of the first layer couples to an adhesive that is configured to couple the first layer to a patient's skin;
a second layer including first and second opposing surfaces, wherein: (a) the first surface of the second layer is fixedly coupled to the second surface of the first layer, and (b) the second layer is more rigid than the first layer;
a third layer including first and second opposing surfaces, wherein: (a) the first surface of the third layer is fixedly coupled to the second surface of the second layer, (b) the third layer is less rigid than the second layer, (c) the second surface of the third layer couples to an adhesive that is configured to couple to the IV line, (d) the second surface of the third layer includes a channel configured to receive the IV line, (e) the first surface of the third layer is primarily disposed in a first plane, and (f) a second plane, parallel to the first plane, intersects the third layer and the channel.

2. The IV line securement system of claim 1, wherein:
a second axis, orthogonal to the first plane, intersects the first and second layers but not the third layer;
a third axis, orthogonal to the first plane, intersects the first, second, and third layers.

3. The IV line securement system of claim 2, wherein:
in a system open orientation the third axis intersects the first layer only one time;
in a system closed orientation the third axis intersects the first layer two times respectively at first and second locations;
the second and third layers are between the first and second locations.

4. The IV line securement system of claim 3, wherein the first and second locations are included on a monolithic portion of the first layer.

5. The IV line securement system of claim 3 comprising at least one compliant pad connected to the first layer, wherein in the system closed orientation the at least one compliant pad and the third layer are configured to sandwich a portion of the IV line between the at least one compliant pad and the third layer.

6. The IV line securement system of claim 2, wherein:
a fourth axis (114), orthogonal to the first plane, intersects the first, second, and third layers and the adhesive coupled to the second surface of the third layer;
in a system open orientation the fourth axis intersects the first layer only one time;
in a system closed orientation the fourth axis intersects the first layer two times respectively at first and second locations;
the second and third layers are between the first and second locations.

7. The IV line securement system of claim 6, wherein the first and second locations are included on a monolithic portion of the first layer.

8. The IV line securement system of claim 2, wherein a first axis, orthogonal to the first plane, intersects the first layer but not the second layer and not the third layer.

9. The IV line securement system of claim 2, wherein:
a portion of the second surface of the second layer is not covered by the third layer;
a portion of the second surface of the third layer is not covered by the adhesive on the second surface of the third layer;
collectively the portions of the second surfaces of each of the second and third layers form a stair-step profile.

10. The IV line securement system of claim 9, wherein:
a fifth axis intersects each of the portions of the second surfaces of each of the second and third layers;
the fifth axis intersects the first plane at an angle of between 0 to 20 degrees.

11. The IV line securement system of claim 1, wherein:
the first layer includes no sublayers;
the second layer includes no sublayers; and
the third layer includes no sublayers.

12. An intravenous (IV) line securement system comprising:
a first layer including first and second opposing surfaces, wherein the first surface of the first layer couples to an adhesive that is configured to couple the first layer to a patient's skin;
a second layer including first and second opposing surfaces, wherein the first surface of the second layer is fixedly coupled to the second surface of the first layer;
a third layer including first and second opposing surfaces, wherein: (a) the first surface of the third layer is fixedly coupled to the second surface of the second layer, (b) the second surface of the third layer couples to an adhesive that is configured to couple to the IV line, (c) the second surface of the third layer includes a channel configured to receive the IV line, (d) the first surface of the third layer is primarily disposed in a first plane, and (e) a second plane, parallel to the first plane, intersects the third layer and the channel.

13. The IV line securement system of claim 12, wherein:
a second axis, orthogonal to the first plane, intersects the first and second layers but not the third layer;
a third axis, orthogonal to the first plane, intersects the first, second, and third layers.

14. The IV line securement system of claim 13, wherein:
in a system open orientation the third axis intersects the first layer only one time;
in a system closed orientation the third axis intersects the first layer two times respectively at first and second locations;
the second and third layers are between the first and second locations.

15. The IV line securement system of claim 14 comprising a compliant pad connected to the first layer at the second location.

16. The IV line securement system of claim 14, wherein the first and second locations are included on a monolithic portion of the first layer.

17. The IV line securement system of claim 13, wherein a first axis, orthogonal to the first plane, intersects the first layer but not the second layer and not the third layer.

18. The IV line securement system of claim 13, wherein:
a fourth axis, orthogonal to the first plane, intersects the first, second, and third layers and the adhesive coupled to the second surface of the third layer;
in a system open orientation the fourth axis intersects the first layer only one time;
in a system closed orientation the fourth axis intersects the first layer two times respectively at first and second locations;
the second and third layers are between the first and second locations.

19. The IV line securement system of claim 18, wherein the first and second locations are included on a monolithic portion of the first layer.

20. The IV line securement system of claim 13, wherein:
a portion of the second surface of the second layer is not covered by the third layer;
a portion of the second surface of the third layer is not covered by the adhesive on the second surface of the third layer;
collectively the portions of the second surfaces of each of the second and third layers form a stair-step profile.

21. The IV line securement system of claim 20, wherein:
a fifth axis intersects each of the portions of the second surfaces of each of the second and third layers;
the fifth axis intersects the first plane at an angle of between 0 to 20 degrees.

22. The IV line securement system of claim 12, wherein:
the first layer includes no sublayers;
the second layer includes no sublayers; and
the third layer includes no sublayers.
